SEQUENCE LISTING (8 sequences)

SEQ ID NO: 1 - DNA, 620 bp, Artificial Sequence
  Target gene sequence ssu16s obtained from Bacillus velezensis ABN1001
gatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcg

SEQ ID NO: 2 - DNA, 448 bp, Artificial Sequence
  Target gene sequence gyrA obtained from Bacillus velezensis ABN1001
atgagcgttatcgtatcccgggcgcttccggatgtgcgtgacggtctgaagccggttcacaggcggattttgtacgcaatgaatgatttaggcatgaccagtgacaaaccatataaaaaatctgcccgtatcgtcggtgaagttatcggtaagtaccacccgcacggtgactcagcggtttacgaatcaatggtcagaatggcgcaggattttaactaccgctacatgcttgttgacggacacggcaacttcggttcggttgacggcgactcagcggccgcgatgcgttacacagaagcgagaatgtcaaaaatcgcaatggaaatcctccgggacattacgaaagatacgattgattatcaagataactatgacggcgcagaaagagaacctgtcgtcatgccttcgagatttccgaatctgctcgtaaacggagctgccggtattg

SEQ ID NO: 3 - DNA, 512 bp, Artificial Sequence
  Target gene sequence gyrB obtained from Bacillus velezensis ABN1001
gacggaaaaaaaaactatcaggcgtacgagcgcggtgtacctgtggccgatcttgaagtgatcggtgatactgataagaccggaacgattacgcacttcgttccggatccggaaattttcaaagaaacaaccgaatacgactatgacctgctttcaaaccgtgtccgggaattggccttcctgacaaaaggtgtaaacatcacgattgaagacaaacgtgaaggacaagaacggaaaaacgagtaccactacgaaggcggaatcaaaagctatgttgagtacttaaaccgttccaaagaagtcgttcatgaagagccgatttatatcgaaggcgagaaagacggcataacggttgaagttgcattgcaatacaacgacagctatacaagcaatatttattctttcacaaataatatcaacacatacgaaggcggcacgcacgaagccggatttaaaaccggtctgacccgtgttataaacgactatgcaagaagaaaagggattttcaaaga

SEQ ID NO: 4 - DNA, 698 bp, Artificial Sequence
  Target gene sequence phoR obtained from Bacillus velezensis ABN1001
ttttttaatttccgtcatatcgtgaaagacgaggacgatgcccttccattcatcgtcggggcccatgatcggaacgccgtcaacttcaaagtagcgccgctcaatattgatgggaagtctgagcaactggcattttttcgtctccgtcataaaaatgtcttcgacaagccgaatgatctcctcatgttcaaacgcatcatggtaaaggcgccgaagcagacgttcaggctttgtatgaaactgcttcgtatatgaccggttcacgagattgataaaacctctcccgtctattaaaatcaaaccggatccgatattttcaataacggttagcagacgatcgcgctgcatatcctgcgttctcgtcatttccattaaatcgaccgccaggctgttcatcgcacgcccgagacggtccgaccgccttgcgtagccgctgtaggagcgggcgtcgtaattccctttggacagctccgtcgccacctttgtcgcggcgtcgattgattttttgtaacgggacgtcatgtttgtataaaagaaaacgataatgataaaagcggtacaaagacttgctgccagcatgccccacatttctccggtaacacttgaaccgccgttgatctcagaggaaacgagtacataccccgcaattttcccggcgtcattttttacggcagtgccgcggatgactttgtttttcg

SEQ ID NO: 5 - DNA, 747 bp, Artificial Sequence
  Target gene sequence groEL obtained from Bacillus velezensis ABN1001
tccttacatggtgactgactctgataagatggaagcggttcttgacaatccttacatcttaatcacagacaaaaaaatcacaaacattcaagaaatccttcctgtgcttgagcaagttgtacagcaaggcaaaccattgcttctgatcgctgaagatgttgaaggggaagctcttgctacactcgttgtcaacaaacttcgcggcacattcaacgctgttgccgttaaagctcctggcttcggtgaccgccgtaaagcaatgcttgaagacatctctgttcttacaggcggagaagtgatcacagaagacttaggccttgacctgaaatctactgaaatcggacaattgggacgcgcttctaaagttgtggtaacgaaagaaaacacaacaatcgtagaaggcgccggcgacactgaaaaaattgctgcacgcgtcaaccaaatccgcgctcaagtggaagaaacaacttctgaattcgacagagaaaaattacaagagcgtcttgcgaaacttgccggcggcgtagctgtcatcaaagtcggcgctgcgactgaaactgagctgaaagagcgtaaacttcgcatcgaagacgccctcaactcaactcgcgcagctgttgaagaaggcatcgtatccggcggtggtacagcgcttgtcaacgtatacaacaaagtcgctgcagtggaagctgaaggcgatgcgcaaacaggtatcaacattgtgcttcgcgcgcttgaagagccgat

SEQ ID NO: 6 - DNA, 697 bp, Artificial Sequence
  Target gene sequence purH obtained from Bacillus velezensis ABN1001
ttgcccccattgaccttgtggtcgtcaacctttacccgtttaaagaaacgatttcaaaagaagacgtaacatacgatgaagcgatagaaaacattgatatcggcggtcccggcatgctgcgcgccgcctcgaaaaaccatcaggatgtgacggtcatcacagatccggccgattacagttccgtgctcaatgagattaaagaacacggcggcgtttctcttaaaagaaaacgcgagcttgcggccaaagtattccgccataccgcggcatacgacgcattaatcgctgattacttaacacgcgaggccgatgagaaagaccctgagcaattcaccgttacatttgagaaaaaacaatcgctccgctacggtgaaaaccctcaccaagaggcggttttctaccaaagcgcacttcccgtctccggttccatcgcggcggcaaaacagcttcacggcaaagagctttcttacaacaatattaaggacgcagatgcggccgttcaaatcgtccgggaatttacagaacccgcagctgttgccgttaaacatatgaacccgtgcggagtcggtacgggagcttcaattgagaagcattcaataaagcgtatgaagctgataaacctccattttcgcggcatcatcgcgctgaaccgtgagttgatcagcacggctgagcccttcacggcatcttttagaat

SEQ ID NO: 7 - DNA, 757 bp, Artificial Sequence
  Target gene sequence rpoB obtained from Bacillus velezensis ABN1001
cacgtgatacaaagcttgggcctgaagagatcacccgcgatattccaaacgtaggggaagacgcgcttcgcaatcttgatgaccgcggaattatccgtatcggtgcggaagtcaacgacggagaccttctcgtaggtaaagtaacgcctaaaggtgtaactgagcttacggctgaagaacgccttctgcatgcgatctttggagaaaaagcgcgtgaagtccgtgatacttctctccgtgtgcctcacggcggcggcggaattatccacgacgtaaaagtcttcaaccgtgaagacggcgacgaacttcctccgggagtgaaccagcttgtacgcgtatatatcgttcagaaacgtaagatttctgaaggtgataaaatggccggacgtcacggaaacaaaggggttatctcgaagattcttcctgaagaagatatgccttaccttcctgacggcacgccgatcgatatcatgcttaacccgctgggtgtaccatcacgtatgaatatcggtcaggtattagaacttcacatgggtatggctgcccgctacctcggcattcacatcgcgtcacctgtatttgacggcgcgcgtgaagaagatgtgtgggaaacacttgaagaagcaggcatgtcaagagacgctaaaacagttctttatgacggccgtacgggagaaccgtttgacaaccgtgtatctgtcggaatcatgtacatgatcaaactggcgcacatggttgatgataaacttcatgcccg

SEQ ID NO: 8 - DNA, 781 bp, Artificial Sequence
  Target gene sequence polC obtained from Bacillus velezensis ABN1001
tcttttgaacggaaaaagcgaaaaatccggtcattgatacgctggaactcgcgcgtttcctgtatcctgagtttaaaaatcaccgcttaaatacgttatgtaagaagtttgatatcgaattaacccagcatcaccgagcggtctttgacgctgaagcaacgggctacctgctgttgaaaatgctcaaagatgccgctgaaaaagacattttttatcatgatcagctgaatgagaatatgggacaatccaatgcttatcagagatcaagaccttatcacgctacattgcttgccgtgaatgagaccggccttaaaaatctgtttaagctcgtgtccatttctcatattcaatatttctacagagtgccgcgcattccgaggtcgcagcttaataaatacagagaaggtctgttaatcggctctgcctgtgacaggggtgaggtctttgaaggcatgatgcaaaaatctcctgaagaggttgaagatatcgcatccttctatgattatcttgaagtgcagccgccggaagtatacagacaccttctgcagcttgagctcgtccgagatgaaaaagcgctgaaagaaatcatcgccaacattacgaagctcggagaaaaattgaataagccggtcgtggctacgggaaatgtccactatttaaacgatgaggataaaatttaccggaagatcttaatatcttcccaaggcggcgccaacccgttaaacagacacgaactgcctaaagtgcacttcagaacgacagacgaaaaatgctttgaaaa

(19) United States
(12) Patent Application Publication
(10) Pub. No.: US 2023/0265380 A1
(43) Pub. Date: Aug. 24, 2023

(54) PLANT GROWTH PROMOTING BACTERIA

(71) Applicants: ABNATURA INC., Trois-Rivières (CA); AUBURN UNIVERSITY, Auburn, AL (US)

(72) Inventors: Jean-Marc JUTEAU, Blainville (CA); Marc SIROIS, Trois Rivières (CA); Joe KLOEPPER, Fort Collins, CO (US); John MCINROY, Auburn, AL (US)

(21) Appl. No.: 18/041,033

(22) PCT Filed: Aug. 10, 2021
(86) PCT No.: PCT/CA2021/051105

§ 371 Date: Feb. 8, 2023

Related U.S. Application Data
(60) Provisional application No. 63/063,649, filed on Aug. 10, 2020.

(51) Int. Cl. C12N 1/20; A01N 63/22

(52) U.S. Cl. CPC C12N 1/205; A01N 63/22; C12R 2001/07

(57) ABSTRACT

A novel bacteria strain Bacillus velezensis ABN1001 was deposited at the International Depositary Authority of Canada. The bacteria, or mutants thereof, or metabolites produced by the bacteria, can be used in compositions for controlling plant disease, treating plant disease, and/or promoting plant growth.

This application claims priority to U.S. Provisional Application No. 63/063,649, filed on Aug. 10, 2020, which is incorporated herein by reference in its entirety.

### TECHNICAL FIELD

The present invention relates to a novel bacteria for promoting plant growth and for use as biopesticide.

### BACKGROUND OF THE ART

There is a need for improvements in agriculture productivity because of a growing world population. The agriculture field is under pressure to produce more from less land. Pests in the present application refer to microorganisms that are a major cause for the loss of productivity in agriculture around the world. In the last forty years, synthetic chemical pesticides for pest control have been responsible for the increase in food production and productivity. However, the use of such chemicals is not a sustainable option for the future of agriculture.

The United States Environmental Protection Agency (EPA) defines biopesticides as a substance, or microorganisms, or a pesticidal substance produced by plants containing added genetic material, that control pests. Biopesticides are generally safer, more biodegradable, and can be less expensive to develop than synthetic chemical pesticides. In addition, pathogens have shown their capacity to develop resistance to synthetic chemical pesticides. There remains concerns over the adverse effects of synthetic pesticides on the environment and on human health. Due to the issues regarding the safety and sustainability of synthetic chemical pesticides, the field of agriculture is looking for alternatives such as biopesticides.

Many of the currently available biopesticides only target a simple major pest. Contans®, based on the fungus Coniothyrium minitans, targets only a single pathogen genus: Sclerotinia. Bioshield™, based on the bacterium Serratia entomophila, controls only a single insect pest. Other biopesticides on the market protect against multiple pests. Serenade®, provides protection for multiple fungal diseases. Chontrol® and Sarritor®, based on Chondrosterum purpureum and Sclerotinia minor respectively, target multiple weed species.

Bacillus bacteria have been investigated for their pathogenic relationships in plant disease protection (Castagnola, A.; Stock, S. P. Common virulence factors and tissue targets of entomopathogenic bacterial for biological control of Lepidopteran pests. Insects 2014, 5, 139-166). In fact, U.S. Pat. Nos. 7,094,592 and 6,077,506 relate to novel bacteria of the Bacillus genus, respectively Bacillus sp. D747 and Bacillus thuringiensis AQ52. Bacillus sp. D747 was identified to be a strain that exhibits effects of controlling several varieties of plant disease and pests without harming plant growth. Bacillus thuringiensis AQ52 was identified to be a novel antibiotic producing strain that demonstrates broad fungicidal and bactericidal activity.

Plant-growth-promoting rhizobacteria (PGPR) are known to be an efficient and environment-friendly alternative to chemical pesticides and fertilizers. Endospore forming bacilli are PGPRs that demonstrate similar long term stability to that of agrochemicals. The endospore forming bacilli are therefore performant biofertilizers. For example, Bacillus amyloliquefaciens FZB42 and has been commercialized by ABiTEP Gmbh as biofertilizer.

### SUMMARY OF THE INVENTION

In one aspect of the present invention, there is provided a novel bacterial strain Bacillus velezensis ABN1001.

In accordance with another aspect of the present invention, there is provided an agent for controlling plant disease comprising Bacillus velezensis ABN1001 bacteria, spores or metabolites obtained from a conditioned culture media of Bacillus velezensis ABN1001.

In accordance with another aspect of the present invention, there is provided an agent for promoting plant growth comprising Bacillus velezensis ABN1001 bacteria, spores or metabolites obtained from a conditioned culture media of Bacillus velezensis ABN1001.

In accordance with another aspect of the present invention, there is provided a method to control plant disease using an agent comprising Bacillus velezensis ABN1001 bacteria, spores or metabolites obtained from a conditioned culture media of Bacillus velezensis ABN1001.

In accordance with another aspect of the present invention, there is provided a method to promote plant growth using an agent comprising Bacillus velezensis ABN1001 bacteria, spores or metabolites obtained from a conditioned culture media of Bacillus velezensis ABN1001.

Many further features and combinations thereof concerning the present improvements will appear to those skilled in the art following a reading of the instant disclosure.

### Definitions

The term “whole broth culture” refers to a solution of liquid culture comprising both cells and media. The term “supernatant” or“conditioned culture media” refers herein to a culture media used to culture a bacteria but from which the bacteria and spores have been removed.

The term “biopesticide” refers herein to a pesticide containing a microorganism. The term “pathogen” refers to microorganisms that are harmful for plants.

### DETAILED DESCRIPTION OF THE INVENTION

The strain Bacillus velezensis ABN1001 was isolated from young corn plant (10 to 17 after planting) The root sample was subjected to vigorous shaking to remove most of the freely attached soil (although soil was still noticeably present). The root sample was then shaken in sterile water to solubilize the bacteria on the root surface (and the closely-associated rhizosphere). The solution was then subjected to serial dilutions with sterile water and then pasteurized (80° C. for 20 minutes) in order to make spore-forming bacteria the predominant type in the collection, but not to necessarily eliminate all others. The dilutions were then spread-plated for colony selection at 2 and again at 5 days. ABN1001 was singled out of 10-100 colonies. The cultures from the initial colony selection was maintained at −80° C. in tryptic soy broth amended with 30% glycerol.

An initial growth promotion assay was performed to quickly screen the bacterial strains. The initial growth promotion assay consisted of a quick screen on soy bean in a greenhouse. Five replicate plants were treated with a 10⁷ cell suspension of the overnight bacterial growth in water and compared to five replicate controls treated with water only. Parameters were measured after about three to five weeks depending on the season the assay is performed in. The initial growth promotion assay demonstrated that Bacillus velezensis ABN1001 promotes plant growth.

Molecular identification of Bacillus velezensis Bacillus velezensis ABN1001 was performed by sequencing the target genes of Bacillus that are known to be plant-growth promoting: ssu16s, gyrA, gyrB, phoR, groEL, purH, rpoB and polC. The sequences were analyzed using the software Basic Local Alignment Search Tool (BLAST).

The target gene ssu16s was found to have sequence identity to ssu16s of Bacillus amyloliquefaciens strain YP6, Bacillus amyloliquefaciens strain BA17 and Bacillus velezensis strain MH25. The target gene sequence ssu16s obtained from Bacillus velezensis ABN1001 is the following:

| | |
|---|---|
| gatgcgtagc cgacctgaga gggtgatcgg ccacactggg actgagacac ggcccagact | 60 |
| cctacgggag gcagcagtag ggaatcttcc gcaatggacg aaagtctgac ggagcaacgc | 120 |
| cgcgtgagtg atgaaggttt tcggatcgta aagctctgtt gttagggaag aacaagtgcc | 180 |
| gttcaaatag ggcggcacct tgacggtacc taaccagaaa gccacggcta actacgtgcc | 240 |
| agcagccgcg gtaatacgta ggtggcaagc gttgtccgga attattgggc gtaaagggct | 300 |
| cgcaggcggt ttcttaagtc tgatgtgaaa gcccccggct caaccgggga gggtcattgg | 360 |
| aaactgggga acttgagtgc agaagaggag agtggaattc cacgtgtagc ggtgaaatgc | 420 |
| gtagagatgt ggaggaacac cagtggcgaa ggcgactctc tggtctgtaa ctgacgctga | 480 |
| ggagcgaaag cgtggggagc gaacaggatt agataccctg gtagtccacg ccgtaaacga | 540 |
| tgagtgctaa gtgttagggg gtttccgccc cttagtgctg cagctaacgc attaagcact | 600 |
| ccgcctgggg agtacggtcg | 620 |

The target gene gyrA was found to have sequence identity to Bacillus velezensis strain JT3-1, Bacillus velezensis strain ZF2 and Bacillus velezensis strain LDO2. The target gene sequence gyrA obtained from Bacillus velezensis ABN1001 is the following:

| | |
|---|---|
| atgagcgtta tcgtatcccg ggcgcttccg gatgtgcgtg acggtctgaa gccggttcac | 60 |
| aggcggattt tgtacgcaat gaatgattta ggcatgacca gtgacaaacc atataaaaaa | 120 |
| tctgcccgta tcgtcggtga agttatcggt aagtaccacc cgcacggtga ctcagcggtt | 180 |
| tacgaatcaa tggtcagaat ggcgcaggat tttaactacc gctacatgct tgttgacgga | 240 |
| cacggcaact tcggttcggt tgacggcgac tcagcggccg cgatgcgtta cacagaagcg | 300 |
| agaatgtcaa aaatcgcaat ggaaatcctc cgggacatta cgaaagatac gattgattat | 360 |
| caagataact atgacggcgc agaaagagaa cctgtcgtca tgccttcgag atttccgaat | 420 |
| ctgctcgtaa acggagctgc cggtattg | 448 |

The target gene gyrB was found to have sequence identity to Bacillus velezensis strain JT3-1, Bacillus velezensis strain ZF2 and Bacillus velezensis strain A2. The target gene sequence gyrB obtained from Bacillus velezensis ABN1001 is the following:

| | |
|---|---|
| gacggaaaaa aaaactatca ggcgtacgag cgcggtgtac ctgtggccga tcttgaagtg | 60 |
| atcggtgata ctgataagac cggaacgatt acgcacttcg ttccggatcc ggaaattttc | 120 |
| aaagaaacaa ccgaatacga ctatgacctg ctttcaaacc gtgtccggga attggccttc | 180 |
| ctgacaaaag gtgtaaacat cacgattgaa gacaaacgtg aaggacaaga acggaaaaac | 240 |
| gagtaccact acgaaggcgg aatcaaaagc tatgttgagt acttaaaccg ttccaaagaa | 300 |
| gtcgttcatg aagagccgat ttatatcgaa ggcgagaaag acggcataac ggttgaagtt | 360 |
| gcattgcaat acaacgacag ctatacaagc aatatttatt ctttcacaaa taatatcaac | 420 |
| acatacgaag gcggcacgca cgaagccgga tttaaaaccg gtctgacccg tgttataaac | 480 |
| gactatgcaa gaagaaaagg gattttcaaa ga | 512 |

The target gene phoR was found to have sequence identity to Bacillus velezensis strain JT3-1, Bacillus velezensis strain ZF2 and Bacillus velezensis strain LDO2. The target gene sequence phoR obtained from Bacillus velezensis ABN1001 is the following:

| | |
|---|---|
| ttttttaatt tccgtcatat cgtgaaagac gaggacgatg cccttccatt catcgtcggg | 60 |
| gcccatgatc ggaacgccgt caacttcaaa gtagcgccgc tcaatattga tcggaagtct | 120 |
| gagcaactgg cattttttcg tctccgtcat aaaaatgtct tcgacaagcc gaatgatctc | 180 |
| ctcatgttca aacgcatcat ggtaaaggcg ccgaagcaga cgttcaggct ttgtatgaaa | 240 |
| ctgcttcgta tatgaccggt tcacgagatt gataaaacct ctcccgtcta ttaaaatcaa | 300 |
| accggatccg atattttcaa taacggttag cagacgatcg cgctgcatat cctgcgttct | 360 |
| cgtcatttcc attaaatcga ccgccaggct gttcatcgca cgcccgagac ggtccgaccg | 420 |
| ccttgcgtag ccgctgtagg agcgggcgtc gtaattccct ttggacagct ccgtcgccac | 480 |
| ctttgtcgcg gcgtcgattg attttttgta acgggacgtc atgtttgtat aaaagaaaac | 540 |
| gataatgata aaagcggtac aaagacttgc tgccagcatg ccccacattt ctccggtaac | 600 |
| acttgaaccg ccgttgatct cagaggaaac gagtacatac cccgcaattt tcccggcgtc | 660 |
| attttttacg gcagtgccgc ggatgacttt gtttttcg | 698 |

The target gene groEL was found to have sequence identity to Bacillus velezensis strain JT3-1, Bacillus velezensis strain ZF2 and Bacillus velezensis strain LDO2. The target gene sequence groEL obtained from Bacillus velezensis ABN1001 is the following:

| | |
|---|---|
| tccttacatg gtgactgact ctgataagat ggaagcggtt cttgacaatc cttacatctt | 60 |
| aatcacagac aaaaaaatca caaacattca agaaatcctt cctgtgcttg agcaagttgt | 120 |
| acagcaaggc aaaccattgc ttctgatcgc tgaagatgtt gaaggggaag ctcttgctac | 180 |
| actcgttgtc aacaaacttc gcggcacatt caacgctgtt gccgttaaag ctcctggctt | 240 |
| cggtgaccgc cgtaaagcaa tgcttgaaga catctctgtt cttacaggcg gagaagtgat | 300 |
| cacagaagac ttaggccttg acctgaaatc tactgaaatc ggacaattgg gacgcgcttc | 360 |
| taaagttgtg gtaacgaaag aaaacacaac aatcgtagaa ggcgccggcg acactgaaaa | 420 |
| aattgctgca cgcgtcaacc aaatccgcgc tcaagtggaa gaaacaactt ctgaattcga | 480 |
| cagagaaaaa ttacaagagc gtcttgcgaa acttgccggc ggcgtagctg tcatcaaagt | 540 |
| cggcgctgcg actgaaactg agctgaaaga gcgtaaactt cgcatcgaag acgccctcaa | 600 |
| ctcaactcgc gcagctgttg aagaaggcat cgtatccggc ggtggtacag cgcttgtcaa | 660 |
| cgtatacaac aaagtcgctg cagtggaagc tgaaggcgat gcgcaaacag gtatcaacat | 720 |
| tgtgcttcgc gcgcttgaag agccgat | 747 |

The target gene purH was found to have sequence identity to Bacillus velezensis strain JT3-1, Bacillus velezensis strain ZF2 and Bacillus velezensis strain LDO2. The target gene sequence purH obtained from Bacillus velezensis ABN1001 is the following:

| | |
|---|---|
| ttgcccccat tgaccttgtg gtcgtcaacc tttacccgtt taaagaaacg atttcaaaag | 60 |
| aagacgtaac atacgatgaa gcgatagaaa acattgatat cggcggtccc ggcatgctgc | 120 |
| gcgccgcctc gaaaaaccat caggatgtga cggtcatcac agatccggcc gattacagtt | 180 |
| ccgtgctcaa tgagattaaa gaacacggcg gcgtttctct taaaagaaaa cgcgagcttg | 240 |
| cggccaaagt attccgccat accgcggcat acgacgcatt aatcgctgat tacttaacac | 300 |
| gcgaggccga tgagaaagac cctgagcaat tcaccgttac atttgagaaa aaacaatcgc | 360 |
| tccgctacgg tgaaaaccct caccaagagg ccgttttcta ccaaagcgca cttcccgtct | 420 |
| ccggttccat cgcggcggca aaacagcttc acggcaaaga gctttcttac aacaatatta | 480 |
| aggacgcaga tgcggccgtt caaatcgtcc gggaatttac agaacccgca gctgttgccg | 540 |
| ttaaacatat gaacccgtgc ggagtcggta cgggagcttc aattgagaag cattcaataa | 600 |
| agcgtatgaa gctgataaac ctccattttc gcggcatcat cgcgctgaac cgtgagttga | 660 |
| tcagcacggc tgagcccttc acggcatctt ttagaat | 697 |

The target gene rpoB was found to have sequence identity to Bacillus velezensis strain JT3-1, Bacillus velezensis strain ZF2 and Bacillus velezensis strain LDO2. The target gene sequence rpoB obtained from Bacillus velezensis ABN1001 is the following:

| | |
|---|---|
| cacgtgatac aaagcttggg cctgaagaga tcacccgcga tattccaaac gtaggggaag | 60 |
| acgcgcttcg caatcttgat gaccgcggaa ttatccgtat cggtgcggaa gtcaacgacg | 120 |
| gagaccttct cgtaggtaaa gtaacgccta aaggtgtaac tgagcttacg gctgaagaac | 180 |
| gccttctgca tgcgatcttt ggagaaaaag cgcgtgaagt ccgtgatact tctctccgtg | 240 |
| tgcctcacgg cggcggcgga attatccacg acgtaaaagt cttcaaccgt gaagacggcg | 300 |
| acgaacttcc tccgggagtg aaccagcttg tacgcgtata tatcgttcag aaacgtaaga | 360 |
| tttctgaagg tgataaaatg gccggacgtc acggaaacaa aggggttatc tcgaagattc | 420 |
| ttcctgaaga agatatgcct taccttcctg acggcacgcc gatcgatatc atgcttaacc | 480 |
| cgctgggtgt accatcacgt atgaatatcg gtcaggtatt agaacttcac atgggtatgg | 540 |
| ctgcccgcta cctcggcatt cacatcgcgt cacctgtatt tgacggcgcg cgtgaagaag | 600 |
| atgtgtggga aacacttgaa gaagcaggca tgtcaagaga cgctaaaaca gttctttatg | 660 |
| acggccgtac gggagaaccg tttgacaacc gtgtatctgt cggaatcatg tacatgatca | 720 |
| aactggcgca catggttgat gataaacttc atgcccg | 757 |

Finally, the target gene polC was found to have sequence identity to Bacillus velezensis strain JT3-1, Bacillus velezensis strain ZF2 and Bacillus velezensis strain LDO2. The target gene sequence polC obtained from Bacillus velezensis ABN1001 is the following:

| | |
|---|---|
| tcttttgaac ggaaaaagcg aaaaatccgg tcattgatac gctggaactc gcgcgtttcc | 60 |
| tgtatcctga gtttaaaaat caccgcttaa atacgttatg taagaagttt gatatcgaat | 120 |
| taacccagca tcaccgagcg gtctttgacg ctgaagcaac gggctacctg ctgttgaaaa | 180 |
| tgctcaaaga tgccgctgaa aaagacattt tttatcatga tcagctgaat gagaatatgg | 240 |
| gacaatccaa tgcttatcag agatcaagac cttatcacgc tacattgctt gccgtgaatg | 300 |
| agaccggcct taaaaatctg tttaagctcg tgtccatttc tcatattcaa tatttctaca | 360 |
| gagtgccgcg cattccgagg tcgcagctta ataaatacag agaaggtctg ttaatcggct | 420 |
| ctgcctgtga caggggtgag gtctttgaag gcatgatgca aaaatctcct gaagaggttg | 480 |
| aagatatcgc atccttctat gattatcttg aagtgcagcc gccggaagta tacagacacc | 540 |
| ttctgcagct tgagctcgtc cgagatgaaa aagcgctgaa agaaatcatc gccaacatta | 600 |
| cgaagctcgg agaaaaattg aataagccgg tcgtggctac gggaaatgtc cactatttaa | 660 |
| acgatgagga taaaatttac cggaagatct taatatcttc ccaaggcggc gccaacccgt | 720 |
| taaacagaca cgaactgcct aaagtgcact tcagaacgac agacgaaaaa tgctttgaaa | 780 |
| a | 781 |

The molecular identification revealed that ABN1001 is a novel bacteria of the genus Bacillus and was named Bacillus velezensis ABN1001.

The morphology of Bacillus velezensis ABN1001 was compared to Bacillus velezensis and Bacillus amyloliquefaciens. This approach revealed rapid and abundant growth of Bacillus velezensis ABN1001 in aerobic conditions (about 18 h) and slow growth in anaerobic conditions (about 72 to 96 h) on agar Brain Heart Infusion (BHI). Bacillus velezensis ABN1001 was found to be mobile in wet mount. Bacillus velezensis ABN1001 is gram positive, has colonies that are of medium size (about 3 mm on BHI agar after 24 hr), have an irregular shape, have a white cream color, have a convex shape, and are mucoid. It was further identified that Bacillus velezensis ABN1001 is a sporulating strain (endospores).

Bacillus velezensis ABN1001 grows on cereus selective agar (CSA) media (meat peptone 10.0, meat extract 1.0, D(−)-mannitol 10.0,s chloride 10.0, phenol red 0.025, agar 12.0, final pH 7.1+/−0.2 at 25° C.) and it fermented mannitol.

The freezing protocol consist of starting with a pure culture on a rich agar medium such as BHI agar. Two colonies are used to inoculate a tube containing steril BHI broth and growth for 16-20 hours at 28° C. One ml of the culture is mixed with 1 ml of steril glycerol 30% and let at room temperature for 15 min. 1.8 ml is transferred in a 2 ml cryogenic screwable tube and stored at minus 80° C.

In order to grow back the bacteria from freezing, the tube (or a portion of the tube content taken aseptically) is thawed at room temperature and transferred in a rich medium such as BHI broth or stricken onto a rich agar medium such as BHI agar. The culture is grown overnight at 28° C. Plants can be administered a culture of Bacillus velezensis ABN1001, a bacterial culture thereof supplemented with other ingredients, a pure bacteria isolated from the bacteria culture, a conditioned culture media or an antifungal or antibacterial metabolite produced by Bacillus velezensis ABN1001 in culture (isolated from a conditioned culture media). The plant can be treated directly for example on the roots, stems, leaves, seeds, or into the soil in the vicinity of the plant to be treated.

The agents of the present invention for controlling plant disease and for promoting plant growth comprise the strain of the present invention Bacillus velezensis ABN1001. The strain can be utilised alone or in combination with one or more variants of ABN1001. The variants include but are not limited to spontaneous mutant strains, mutant strains obtained by ultra-violet or chemical mutagen treatment, cell fusion strains, and genetic recombination strains. The culture can be used to create formulations in which the strain is diluted with at least one of an inert liquid or solid carrier, a surfactant, protective agents, and other auxiliary agents if necessary.

The agents of the present invention for controlling plant disease and for promoting plant growth can be utilised alone or in combination with one or more plant growth-promoting bacteria such as Bacillus amyloliquefaciens strains D747, QST713, GB03, MBI600, FZB24, or FZB42, or Bacillus pumilus strains INR7 (also known as GB34) or QST2808.

The agents of the present invention for controlling plant disease and for promoting plant growth can comprise for example wettable powders, dry flowables, microencapsulation agents, liquid or solid formulations, antibiotic extracted from microbial cultures, whole broth culture, conditioned culture media, granules, suspensions, or emulsifiable concentrate. Biopesticides may be applied in combination with one or more chemical pesticide, herbicide, or fungicide.

As known in the art, carriers for the agent of the present invention can comprise, for example, one of porous solid carriers such as talc, bentonite, clay, kaolin, diatomaceous earth, white carbon, vermiculite, slaked lime, siliceous sand, ammonium sulfate, and urea. Liquid carriers can, for example, be one of water, isopropyl alcohol, xylene, cyclohexanone, methylnaphthalene, and alkyl glycol.

As known in the art surfactants and dispersants for the agent of the present invention can comprise, for example, one of dinaphthylmethanesulfonates, alcohol sulfates, alkyl aryl sulfonates, lignin sulfonates, polyoxyethylene glycol ethers, polyoxyethylene alkyl aryl ethers, and polyoxyethylene sorbitan monoakylates.

As known in the art auxiliary agents for the agent of the present invention can comprise, for example, one of carboxymethylcellulose, polyethylene glycol, propylene glycol, gum Arabic, and xanthan gum.

As known in the art auxiliary agents for the agent of the present invention can further comprise, for example, skim milk or pH buffers.

The method to apply biopesticides or biofertilizers on plants is well known in the art. For example, Bacillus velezensis ABN1001, or a composition containing same, can be applied in the form of wettable powders, dry flowables, microencapsulation of agents, liquid or solid formulations, antibiotic extracted from microbial cultures, whole broth culture, granules, suspensions, or emulsifiable concentrate. It may also be applied in combination with one or more chemical pesticide, herbicide, or fungicide.

### Example 1

### Protection Against Plant Disease

Acetoin is a compound that confers plant immunity against a wide range of diseases by activating plant defences against pathogens (Rudrappa, Thimmaraju, et al. “The rhizobacterial elicitor acetoin induces systemic resistance in Arabidopsis thaliana.” Communicative & Integrative Biology 3.2 (2010): 130-138). A colorimetric assay was used to measure the secretion of acetoin of Bacillus velezensis ABN1001 with Enterobacter aerogenes as the positive control. The measurement was performed using the Voges-Proskauer to infer the concentration level from the colorimetric measurement (Westerfeld, W. W. “A colorimetric determination of blood acetoin.” J. biol. Chem 161.2 (1945): 495-502). The concentration level comparisons are summarized in Table 1.

TABLE 1
Results for the production of acetoin
by Bacillus velezensis ABN1001

| Strains | First colorimetric assay |
|---|---|
| Enterobacter aerogenes | +++ |
| Bacillus velezensis | +++ |
| ABN1001 | |

As it is known that acetoin is responsible for conferring protection against a wide range of diseases and as it is also known to promote plant growth, it is understood that Bacillus velezensis ABN1001 also promotes plant growth and confer plant resistance to a wide range of diseases.

### Example 2

### Antifungal Activity Against Trichoderma harzianum T-22

Bacillus velezensis ABN1001 was cultured for 72 hours in yeast extracts-sugar media (20 g/L yeast peptone, 15 g/L molasses, 15 g/L saccharose) and stirred in an Erlenmeyer. A volume of the supernatant of the culture mixture (100 μL) was extracted and tested in tubes placed on a surface of fungus Trichoderma harzianum T-22. The control strain used was Bacillus amyloliquefaciens FZB24. The inhibition zone was measured and the results are summarized in Table 2.

TABLE 2
Results of the antifungal activity of Bacillus velezensis
ABN1001 against Trichoderma harzianum T-22

| | First assay | Second assay |
|---|---|---|
| | Inhibition zone | Inhibition zone |
| Strains | (average in mm) | (average in mm) |
| FZB24 | 20.33 | 17.14 |
| ABN1001 | 14.73 | 19.57 |
| | | Δ = +12% |

The strain of the present invention Bacillus velezensis ABN1001 showed a larger inhibition zone than the control. Therefore, Bacillus velezensis ABN1001 exhibits antifungal activity against Trichoderma harzianum T-22.

### Example 3

### Broad Antifungal Activity

Bacillus velezensis ABN1001 was cultured for 72 hours in yeast extracts-sugar media (20 g/L yeast peptone, 15 g/L molasses, 15 g/L saccharose) and stirred in an Erlenmeyer. A volume of the supernatant of the culture mixture (100 μL) was extracted and tested in tubes (also known as penicylinder) placed on Petri dishes inoculated with Fusarium solani, Botrytis cinerea, Pythium splendens, Colletotrichum acutatum, Rhizoctonia solani, Verticillium dahliae or Sclerotinia sclerotiorum. The positive control strain used was Bacillus amyloliquefaciens FZB24, a commercially available strain isolated from the product FZB24 of the company ABITEP GmbH (Berlin, Germany). The inhibition zone was measured and the results are summarized in Table 3.

TABLE 3
Results for the broad antifungal activity
of Bacillus velezensis ABN1001

| | First assay | Second assay |
|---|---|---|
| | Inhibition zone | Inhibition zone |
| Strains | (average in mm) | (average in mm) |
| FZB24 | 19.86 | 18.72 |
| ABN1001 | 9.22 | 16.74 |
| | Δ = −56% | Δ = −11% |

Therefore, the strain of the present invention Bacillus velezensis ABN1001 has shown antifungal activity comparable to that of the positive control, hence having broad antifungal effects.

### Example 4

### Broad Antibacterial Activity

To demonstrate by way of example, Bacillus velezensis ABN1001 was cultured for 72 hours in yeast extracts-sugar (20 g/L yeast peptone, 15 g/L molasses, 15 g/L saccharose) media and stirred in an Erlenmeyer. A volume of the supernatant of the culture mixture (100 μL) was extracted and tested in tubes (penicylinders) placed on Petri dishes inoculated with containing Streptomyces scabies, Pseudomonas syringae, Clavibacter michiganensis, Xanthomonas campestris, Pseudomonas aeruginosa and Pectobacterium caravoterum. The control strain, as in the previous example, was FZB24. The inhibition zone was measured and the results are summarized in Table 4.

TABLE 4
Results for the broad antibacterial activity
of Bacillus velezensis ABN1001

| | Inhibition zone |
|---|---|
| Strains | (average in mm) |
| FZB24 | 19.61 |
| ABN1001 | 12.76 |

A significant inhibition zone was formed, therefore the strain of the present invention Bacillus velezensis ABN1001 has broad antibacterial effects.

### Example 5

### Sporulation and Antifungal Effect on Fusarium solani after Fermentation

Bacillus velezensis ABN1001 was fermented at maximal agitation rate and aeration in a bioreactor of 150 L. The fermentation lasted 48 hours in a media of Yeast Extracts-Sugar (20 g/L yeast peptone, 15 g/L molasses, 15 g/L saccharose). The process was performed a second time in a bioreactor of 500 L. The spore count results are summarized in Table 5.

TABLE 5
Colony forming units (CFU) of Bacillus velezensis ABN1001
after fermentation at maximal agitation rate and aeration

| | | CFU/mL for | CFU/mL for |
|---|---|---|---|
| | | the 150 L | the 500 L |
| Time | Sample | bioreactor | bioreactor |
| 30 h | Total cells | 5.95 × 10⁹ | 1.05 × 10¹⁰ |
| | Spores | 7 × 10⁹ | 8.73 × 10⁹ |
| 48 h | Total cells | (not measured) | 9.8 × 10⁹ |
| Not Pasteurized | Spores | (not measured) | 9.77 × 10⁹ |
| 48 h | Spores without | 4.4 × 10⁹ | 9.17 × 109 |
| Pasteurized | preservation | | |
| | agents | | |
| | Spores with | 3.13 × 109 | 5.3 × 10⁹ |
| | preservation | | |
| | agents | | |

A volume of the supernatant (not containing cells or spores) of the culture mixture (100 μL) was extracted and tested in tubes placed on a surface containing Fusarium solani diluted at 1/100. In some tests, propionic acid as a preservative agent was added to the supernatant. The inhibition zone was measured and the results are summarized in Table 6.

TABLE 6
Results of the antifungal activity of Bacillus velezensis
ABN1001 against Fusarium solani

| | | | Inhibition zone | Inhibition zone |
|---|---|---|---|---|
| | | Supernatant | (mm) from 150 L | (mm) from 500 L |
| Time | Sample | volume | bioreactor | bioreactor |
| 48 h | Supernatant only | 100 μL | 18.70 | 20.21 |
| Pasteurised | Supernatant only | 200 μL | 20.27 | 22.14 |
| | Supernatant with | 100 μL | 37.12 | 37.22 |
| | preservation | | | |
| | agents | | | |
| | Supernatant with | 200 μL | 43.66 | 48.18 |
| | preservation | | | |
| | agents | | | |

The strain of the present invention Bacillus velezensis ABN1001 has good sporulation efficiency and an antifungal effect on Fusarium solani.

### Example 6

### Production of Antimicrobial Metabolites

Bacillus velezensis ABN1001 was cultured for 72 hours in yeast extracts-sugar media and stirred in an Erlenmeyer. The supernatant was then analysed by liquid chromatography-mass spectrometry (LC-MS) to detect and quantify the lipopeptides surfactin, fengycin and iturin. These lipopeptides are antibacterial, antifungal, and reduce plant disease. Iturin and fengycin exhibit powerful antifungal activity and growth inhibition against other pathogens as well. Surfactins are not toxic for fungal pathogens but have a synergistic effect on the antifungal activity of Iturin. (Kim, Pyoung II, et al. “Production of biosurfactant lipopeptides Iturin A, fengycin and surfactin A from Bacillus subtilis CMB32 for control of Colletotrichum gloeosporioides.” J Microbiol Biotechnol 20.1 (2010): 138-145). The positive control, as in some of the previous examples, was Bacillus amyloliquefaciens FZB24. The results are summarized in Table 7 with the standard deviation (SD).

TABLE 7
Results of the secretion of antimicrobial metabolites
by Bacillus velezensis ABN1001

| | Concentration | Concentration | Concentration |
|---|---|---|---|
| | of Surfactin | of Fengycin | of Iturins |
| Sample | (ppm) ± SD | (ppm) ± SD | (ppm) ± SD |
| ABN1001 | 1388.61 ± 379.14 | 927.47 + 281.14 | 65.03 ± 5.90 |
| FZB24 | 837.27 ± 142.71 | 583.65 ± 183.49 | 11.03 ± 2.43 |

The strain Bacillus velezensis ABN1001 produces metabolites that are antimicrobial as can be seen from Table 7. The concentrations of the studied lipopeptides produced by the culture of strain Bacillus velezensis ABN1001 are greater than the positive control.

### Example 7

### Promoting Growth of Soya

Four assays were performed to assess the soya growth stimulation activity. In the first assay (#1), 12 soya seeds were placed in separate wells with Promix™ soil (Premier Horticulture LTD. Rivère du Loup, QC Canada). The culture was maintained for three weeks with regular watering containing 10⁶ CFU/mL of Bacillus velezensis ABN1001. The control seeds were watered with tap water without adding Bacillus velezensis ABN1001. After three weeks, the plants were weighted and the growth of the roots was assessed visually. The results are summarized in Table 8.

In the second assay (#2), 12 soya seeds were placed in separate wells with Promix™ soil (Premier Horticulture LTD. Rivière du Loup, QC Canada). The culture was maintained for three weeks with regular watering containing 10⁶ CFU/mL of Bacillus velezensis ABN1001. The control seeds were watered with tap water without adding Bacillus velezensis ABN1001. After three weeks, the plants were weighted and the growth of the roots was assessed visually. The results are summarized in Table 8.

In the third assay (#3), 20 soya seeds were placed in separate wells with half Miracle Grow potting soil, half sand. The culture was maintained for four weeks with watering every week containing 10⁵ CFU/mL of Bacillus velezensis ABN1001. The control seeds were watered with tap water without adding Bacillus velezensis ABN1001. After four weeks, the plants were dried then the roots and leafs were weighted. The results are summarized in Table 8.

In the fourth assay (#4), 28 soya seeds were placed in separate wells with half Miracle Grow soil, half sand. The culture was maintained for four weeks with watering every week containing 10⁵ CFU/mL Bacillus velezensis ABN1001. The control seeds were watered with tap water without adding Bacillus velezensis ABN1001. After four weeks, the plants were dried then the roots and leafs were weighted. The nodules were also counted. The results are summarized in Table 8.

TABLE 8
Results of the growth assay on soya

| | Average | | Average | Average | |
|---|---|---|---|---|---|
| Assay | plant | | root | leafs | Average |
| number | weight | Growth | weight | weight | nodule per |
| and sample | (g/plant) | of roots | (g/root) | (g/leafs) | plant |
| #1 Tap | 1.3 | ++ | | | |
| water | | | | | |
| control | | | | | |
| #1 Water | 1.63 | +++ | | | |
| with | Δ = +20% | | | | |
| ABN1001 | | | | | |
| #2 Tap | 1.86 | ++ | | | |
| water | | | | | |
| control | | | | | |
| #2 Water | 1.99 | +++ | | | |
| with | Δ = +7% | | | | |
| ABN1001 | | | | | |
| #3 Tap | | | 0.1469 | 0.59 | |
| water | | | | | |
| control | | | | | |
| #3 Water | | | 0.1738 | 0.74 | |
| with | | | Δ = +15% | Δ = +20% | |
| ABN1001 | | | | | |
| #4 Tap | | | 0.1868 | 0.79 | 7 |
| water | | | | | |
| control | | | | | |
| #4 Water | | | 0.2709 | 1.01 | 15 |
| with | | | Δ = +31% | Δ = +22% | Δ = +47% |
| ABN1001 | | | | | |

As can be observed from the results, the strain of the present invention Bacillus velezensis ABN1001 promotes growth.

### Example 8

### Promoting Growth of Corn

To demonstrate by way of example, four assays were performed to assess the corn growth stimulation activity. In the first assay (#1), 12 corn seeds were placed in separate wells with Miracle Grow potting soil. The culture was maintained for three weeks with regular watering containing 10⁶ CFU/mL of Bacillus velezensis ABN1001. The control seeds were watered with tap water without adding Bacillus velezensis ABN1001. After three weeks, the plants were weighted and the growth of the roots was assessed visually. The results are summarized in Table 9.

In the second assay (#2), 9 corn seeds were placed in separate wells with Promix soil. The culture was maintained for four weeks with regular watering containing 10⁵ CFU/mL of Bacillus velezensis ABN1001. The control seeds were watered with tap water without adding Bacillus velezensis ABN1001. After four weeks, the plants were weighted and the growth of the roots was assessed visually. The results are summarized in Table 9.

In the third assay (#3), 20 corn seeds were placed in separate wells with half Miracle Grow potting soil, half sand. The culture was maintained for four weeks with watering every week containing 10⁵ CFU/mL of Bacillus velezensis ABN1001. The control seeds were watered with tap water without adding Bacillus velezensis ABN1001. After four weeks, the plants were dried then the roots and leafs were weighted. The results are summarized in Table 9.

In the fourth assay (#4), 25 corn seeds were placed in separate wells with half Miracle Grow soil, half sand. The culture was maintained for four weeks with watering every week containing 10⁵ CFU/mL of Bacillus velezensis ABN1001. The control seeds were watered with tap water without adding Bacillus velezensis ABN1001. After four weeks, the plants were dried and the roots and leafs were then weighted. The results are summarized in Table 9.

TABLE 9
Results of the growth assay on corn

| | Average | | Average | Average |
|---|---|---|---|---|
| Assay | plant | | root | leafs |
| number | weight | Growth | weight | weight |
| and sample | (g/plant) | of roots | (g/root) | (g/leafs) |
| #1 Tap | 3 | ++ | | |
| water | | | | |
| control | | | | |
| #1 Water | 4.42 | +++ | | |
| with | Δ = +32% | | | |
| ABN1001 | | | | |
| #2 Tap | 1.39 | ++ | | |
| water | | | | |
| control | | | | |
| #2 Water | 1.81 | +++ | | |
| with | Δ = +23% | | | |
| ABN1001 | | | | |
| #3 Tap | | | 0.2344 | 1.02 |
| water | | | | |
| control | | | | |
| #3 Water | | | 0.3464 | 1.04 |
| with | | | Δ = +22% | Δ = +2% |
| ABN1001 | | | | |
| #4 Tap | | | 0.2105 | 0.74 |
| water | | | | |
| control | | | | |
| #4 Water | | | 0.2659 | 0.9 |
| with | | | Δ = +21% | Δ = +18% |
| ABN1001 | | | | |

As demonstrated by the results, the strain of the present invention Bacillus velezensis ABN1001 promotes growth.

### Example 9

### Growth Promoting Activity on Cucumber and Tomato Plants

In this assay, 25 seeds of each of Gusto cucumber and Sub Artic Plenty tomatoes (McKenzie Seeds, Brandon, Manitoba, Canada) were selected. The seeds were incubated in a solution of 10⁵ Bacillus velezensis ABN1001 per mL for 3 minutes. Then the seeds were seeded in half Miracle Grow potting soil, half sand. The culture was maintained with watering containing 10⁵ CFU/mL of Bacillus velezensis ABN1001 three times per 5-10 days. The control seeds were not incubated in the solution and were watered with tap water without adding Bacillus velezensis ABN1001. The roots were harvested after 55-60 days in culture. The roots were then washed with tap water and left at room temperature for 24 hours. Finally, the roots were placed in brown paper bags inside an incubator at 60° C. for one week and weighted at the end of the week. The results are summarized in Table 10.

TABLE 10
Results of the growth assay for cucumbers and tomatoes

Average weight of the roots (g)

| Sample | Cucumber | Tomato |
|---|---|---|
| Water tap (control) | 0.1391 | 0.2789 |
| Water containing ABN1001 | 0.2037 | 0.3306 |
| | Δ = +32% | Δ = +16% |

The results of Table 10 demonstrate that Bacillus velezensis ABN1001 promotes growth.

### Example 10

### Strawberry Field Trial

Bacillus velezensis ABN1001 in combination Bacillus velezensis ABN110 was tested for activity on disease protection and yield improvement in strawberry field assays. A pasteurized fermentation spore solution was adjusted at a concentration of 2×10⁹ cfu/ml per strain. The treatment consisted of weekly or every two weeks spraying of a solution at 1 liter per hectare. Eight (8) applications during the growing season were applied. Yield data collection was done every week. Disease control was measured at one month before the end of the season by counting powdery mildew affected leaves per plant. As seen in Table 11, results showed a significant improvement in yield and diseases control.

TABLE 11
Results on yield (Increase %) and disease
control on strawberry plants
Yield (Sellable Fruits g/plant)

| Treatment | Control | Increase % | Signification |
|---|---|---|---|
| 91 | 69 | 31 | 99% |

Powdery Mildew Control (Nb Leaves Affected/plant)

| Treatment | Control | Decrease | Signification |
|---|---|---|---|
| 0.8 | 2.95 | −72 | 99% |

### CONCLUSION

The bacterial strain Bacillus velezensis ABN1001 is a novel bacterial strain that exhibits protection against many plant diseases, broad antifungal activity, broad antibacterial activity, and promotes plant growth. Therefore, Bacillus velezensis ABN1001 can be used to protect plants against disease, to treat plant disease, and to promote plant growth.

As can be seen therefore, the examples described above and illustrated are intended to be exemplary only. The scope is indicated by the appended claims.

The strain of the present invention was deposited at the National Microbiology Laboratory, International Depositary Authority of Canada (IDAC), 1015 Arlington Street, Winnipeg, Manitoba, Canada, R3E 3R2, as “Bacillus velezensis ABN1001” with Accession Number 040820-01 on Aug. 4, 2020.

What is claimed is:

1. The bacteria of claim 6, said bacteria being as deposited at IDAC under Accession No. 040820-01, strain “Bacillus velezensis ABN1001”.

2.-5. (canceled)

6. A bacteria having all the identifying characteristics of Bacillus velezensis ABN1001, deposited at IDAC under Accession No. 040820-01 and mutants thereof, said bacteria and mutants having at least one of plant disease protection activity, broad antifungal activity, broad antibacterial activity or plant growth promoting activity.

7. A composition comprising the bacteria as defined in claim 6 and a carrier.

8. The composition of claim 7, wherein the bacteria are present as spores.

9. (canceled)

10. The composition according to claim 0, comprising at least 10⁵ CFU/mL of the bacteria of said composition.

11. The composition according to claim 7, wherein the composition is formulated as a granule, fine powder, wettable powder, dry flowables, microencapsulation of agents, liquid formulation, solid formulation, whole broth culture, suspension concentrate or emulsifiable concentrate.

12. The composition according to claim 7, further comprising a surfactant or a dispersant.

13. The composition according to claim 7, further comprising at least one of a pesticide, fungicide or herbicide.

14. The composition according to claim 7, further comprising at least one of plant growth modifier, fertilizer or manure.

15. A composition comprising a supernatant extracted from a culture of the bacteria according to claim 6.

16. A composition comprising metabolites extracted from a culture of the bacteria according to claim 6.

17. The composition of claim 7, further comprising one or more plant growth-promoting bacteria.

18. The composition of claim 17, wherein the plant growth-promoting bacteria is Bacillus amyloliquefaciens strains D747, QST713, GB03, MBI600, FZB24, or FZB42, or Bacillus pumilus strains INR7 (also known as GB34) or QST2808.

19. A method for treating or protecting plants against disease comprising administering to said plant the bacteria as defined in claim 6.

20. (canceled)

21. A method for promoting plant growth comprising administering to said plant the bacteria as defined in claim 6.

22. The method according to claim 0, wherein said disease is caused by at least one of Streptomyces scabies, Pseudomonas syringae, Clavibacter michiganensis, Xanthomonas campestrs, Pseudomonas aeruginosa, Pectobacterum caravoterum, Fusarium solani, Botrytis cineea, Pythium splendens, Colletotrichum acutatum, Rhizoctonia solani, Verticillium dahliae or Sclerotinia sclerotiorum.

23.-30. (canceled)
